(19) United States
(12) Patent Application Publication
(10) Pub. No.: US 2012/0078282 A1
(43) Pub. Date: Mar. 29, 2012

(54) SCISSOR SYSTEM FOR ENDOSCOPIC AND OPEN SURGICAL PROCEDURE

(76) Inventor: STEVE LIVNEH, Amherstburg (CA)

(21) Appl. No.: 13/247,734

(22) Filed: Sep. 28, 2011

Related U.S. Application Data
(60) Provisional application No. 61/387,076, filed on Sep. 28, 2010.

(51) Int. Cl. A61B 17/32

(52) U.S. Cl. 606/174

(57) ABSTRACT

Scissor systems are described. The scissor systems are provided with a secondary, lower parallel jaw member that supports a stand alone blade or jaw member against a reinforced blade or jaw member continuously through the cutting action, while simultaneously maintaining continuous interference, i.e., shear action, and sharply reducing the probability of skewing or spreading.

### CROSS-REFERENCE TO RELATED APPLICATION

The instant application claims priority to U.S. Provisional Patent Application Ser. No. 61/387,076, filed Sep. 28, 2010, the entire specification of which is expressly incorporated herein by reference.

### FIELD OF INVENTION

The present invention relates generally to scissor systems, and more particularly to scissor systems that improve the performance of curved, straight and hooked scissors, by adding a supporting secondary jaw member such that the characteristics of a typical scissor's skewing or failure to cut due to reduced sharpness or excessive bite are eliminated or at least reduced significantly.

### BACKGROUND OF THE INVENTION

Endoscopy, and especially laparoscopic endoscopy, has been a rapidly growing surgical practice in the past few decades. The technology began, and is still managed, primarily with hand held instruments, the majority of which are instruments using monopolar RF energy sources to assist in coagulation, cauterizing and cutting of various types of tissue. The largest selling type of these instruments is scissors. Scissors of many designs have been designed and applied to endoscopic surgery, such as, but not limited to: (1) double jaw scissors, including straight and curved designs; (2) single jaw scissors, including straight and curved designs; (3) hook scissors, including single jaw and straight designs; and variants of the above.

Surgeons expect scissors to be sharp at all times and to cut for all or at least the majority of the jaws' length. Furthermore, surgeons expect perfect cutting action with both thick and thin tissue, soft and hard tissue, and/or slippery tissue. These expectations present serious challenges to the designers of surgical scissors. Unfortunately, conventional scissor systems, including those used for various surgical applications, do not provide the performance needed by surgeons, especially with respect to providing exceptional and consistent cutting action.

Accordingly, there exists a need for new and improved scissor systems that overcomes at least one of the above-described disadvantages.

### SUMMARY OF THE INVENTION

In accordance with the general teachings of the present invention, new and improved scissor systems are provided. More specifically, the present invention provides new and improved scissor systems that may be used in various surgical applications. By way of a non-limiting example, the present invention provides scissor systems in various combinations wherein continuous cut and non-skewing characteristics are provided even when the scissor is not perfectly sharp and is compatible with a large variety of tissue types, e.g., thin, thick, hard, soft or even slippery. By way of another non-limiting example, these design parameters of the scissor systems of the present invention are achieved, at least in part, by adding to the scissor system's designs a secondary, lower parallel jaw member, thus supporting a stand alone blade or jaw member against the reinforced blade or jaw member continuously through the cutting action, while maintaining continuous interference, i.e., shear action, and sharply reducing the probability of skewing or spreading.

In accordance with one embodiment of the present invention, a scissor system is provided, comprising: a first jaw member having a cutting surface formed thereon; a second jaw member having a cutting surface formed thereon; and a beam member positioned substantially parallel to the cutting surface of the second jaw member.

In accordance with an aspect of this embodiment, the first and second jaw members are pivotally engaged to each other so as to be selectively operable to open apart from each other and close together towards each other to selectively perform a cutting action.

In accordance with an aspect of this embodiment, the beam member maintains engagement of the cutting surfaces of the first and second jaw members through substantially the entirety of the cutting action.

In accordance with an aspect of this embodiment, the beam member supports the first jaw member so as to substantially prevent the first jaw member from being pushed away from the second jaw member during the cutting action.

In accordance with an aspect of this embodiment, either the first or second jaw members include a curved portion.

In accordance with an aspect of this embodiment, the curved portions of the first and second jaw members are not identical.

In accordance with an aspect of this embodiment, the first jaw member includes a hook member formed on a distal portion thereof.

In accordance with an aspect of this embodiment, the second jaw member includes a pair of spaced and opposed hook members formed on a distal portion thereof.

In accordance with an aspect of this embodiment, the second jaw member and the beam member define a cavity formed therebetween.

In accordance with an aspect of this embodiment, the first jaw member is selectively operable to be at least partially received within the cavity.

In accordance with a first alternative embodiment of the present invention, a scissor system is provided, comprising: a first jaw member having a cutting surface formed thereon; a second jaw member having a cutting surface formed thereon; and a beam member positioned substantially parallel to the cutting surface of the second jaw member; wherein the first and second jaw members are pivotally engaged to each other so as to be selectively operable to open apart from each other and close together towards each other to selectively perform a cutting action; wherein the beam member maintains engagement of the cutting surfaces of the first and second jaw members through substantially the entirety of the cutting action.

In accordance with an aspect of this embodiment, the beam member supports the first jaw member so as to substantially prevent the first jaw member from being pushed away from the second jaw member during the cutting action.

In accordance with an aspect of this embodiment, either the first or second jaw members include a curved portion.

In accordance with an aspect of this embodiment, the curved portions of the first and second jaw members are not identical.

In accordance with an aspect of this embodiment, the first jaw member includes a hook member formed on a distal portion thereof.

In accordance with an aspect of this embodiment, the second jaw member includes a pair of spaced and opposed hook members formed on a distal portion thereof.

In accordance with an aspect of this embodiment, the second jaw member and the beam member define a cavity formed therebetween.

In accordance with an aspect of this embodiment, the first jaw member is selectively operable to be at least partially received within the cavity.

In accordance with a second alternative embodiment of the present invention, a scissor system is provided, comprising: a first jaw member having a cutting surface formed thereon; a second jaw member having a cutting surface formed thereon; and a beam member positioned substantially parallel to the cutting surface of the second jaw member; wherein the first and second jaw members are pivotally engaged to each other so as to be selectively operable to open apart from each other and close together towards each other to selectively perform a cutting action; wherein the beam member maintains engagement of the cutting surfaces of the first and second jaw members through substantially the entirety of the cutting action; wherein the beam member supports the first jaw member so as to substantially prevent the first jaw member from being pushed away from the second jaw member during the cutting action.

In accordance with an aspect of this embodiment, either the first or second jaw members include a curved portion.

In accordance with an aspect of this embodiment, the curved portions of the first and second jaw members are not identical.

In accordance with an aspect of this embodiment, the first jaw member includes a hook member formed on a distal portion thereof.

In accordance with an aspect of this embodiment, the second jaw member includes a pair of spaced and opposed hook members formed on a distal portion thereof.

In accordance with an aspect of this embodiment, the second jaw member and the beam member define a cavity formed therebetween.

In accordance with an aspect of this embodiment, the first jaw member is selectively operable to be at least partially received within the cavity.

Further areas of applicability of the present invention will become apparent from the detailed description provided hereinafter. It should be understood that the detailed description and specific examples, while indicating the preferred embodiment of the invention, are intended for purposed of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates a perspective view of a surgical instrument including a scissor system, in accordance with a first embodiment of the present invention;

FIG. 2 illustrates a top perspective view of the scissor system depicted in FIG. 1, in accordance with a first embodiment of the present invention;

FIG. 3 illustrates a bottom perspective view of the scissor system depicted in FIG. 1, in accordance with a first embodiment of the present invention;

FIG. 4 illustrates a second top perspective view of the scissor system depicted in FIG. 1, in accordance with a first embodiment of the present invention;

FIG. 5 illustrates a third top perspective view of the scissor system depicted in FIG. 1, in accordance with a first embodiment of the present invention;

FIG. 6 illustrates a top perspective view of the lower jaw member of the scissor system depicted in FIG. 1, in accordance with a first embodiment of the present invention;

FIG. 7 illustrates a second top perspective view of the lower jaw member of the scissor system depicted in FIG. 1, in accordance with a first embodiment of the present invention;

FIG. 8 illustrates a side view of the upper jaw member of the scissor system depicted in FIG. 1, in accordance with a first embodiment of the present invention;

FIG. 9 illustrates an end view of the scissor system depicted in FIG. 1, in accordance with a first embodiment of the present invention;

FIG. 10 illustrates a perspective view of the activating shaft member inside the hollow tube member of the surgical instrument depicted in FIG. 1, in accordance with a first embodiment of the present invention;

FIG. 11 illustrates a second perspective view of the activating shaft member of the surgical instrument depicted in FIG. 1, in accordance with a first embodiment of the present invention;

FIG. 12 illustrates a perspective view of the hollow tube member of the surgical instrument depicted in FIG. 1, in accordance with a first embodiment of the present invention;

FIG. 13 illustrates a perspective view of the isolative material of the surgical instrument depicted in FIG. 1, in accordance with a first embodiment of the present invention;

FIG. 14 illustrates a perspective view of a first alternative scissor system, in accordance with a second embodiment of the present invention;

FIG. 15 illustrates a second perspective view of the scissor system depicted in FIG. 14, in accordance with a second embodiment of the present invention;

FIG. 16 illustrates a top perspective view of the scissor system depicted in FIG. 14, in accordance with a second embodiment of the present invention;

FIG. 17 illustrates a perspective view of a second alternative scissor system, in accordance with a third embodiment of the present invention;

FIG. 18 illustrates a perspective view of a lower jaw member of the scissor system depicted in FIG. 17, in accordance with a third embodiment of the present invention;

FIG. 19 illustrates a perspective view of an upper jaw member of the scissor system depicted in FIG. 17, in accordance with a third embodiment of the present invention;

FIG. 20 illustrates a perspective view of a third alternative scissor system, in accordance with a fourth embodiment of the present invention;

FIG. 21 illustrates a perspective view of an upper jaw member of the scissor system depicted in FIG. 20, in accordance with a fourth embodiment of the present invention;

FIG. 22 illustrates a perspective view of a lower jaw member of the scissor system depicted in FIG. 20, in accordance with a fourth embodiment of the present invention;

FIG. 23 illustrates a perspective view of a fourth alternative scissor system, in accordance with a fifth embodiment of the present invention;

FIG. 24 illustrates a perspective view of a fifth alternative scissor system, in accordance with a sixth embodiment of the present invention;

FIG. 25 illustrates a perspective view of a sixth alternative scissor system, in accordance with a seventh embodiment of the present invention;

FIG. 26 illustrates a perspective view of the scissor system depicted in FIG. 25 with the hollow tube member removed for purposes of illustration, in accordance with a seventh embodiment of the present invention;

FIG. 27 illustrates a perspective view of the hollow tube member of the scissor system depicted in FIG. 25 with the jaw members removed for purposes of illustration, in accordance with a seventh embodiment of the present invention;

FIG. 28 illustrates a perspective view of the lower jaw member of the scissor system depicted in FIG. 25, in accordance with a seventh embodiment of the present invention;

FIG. 29 illustrates a second perspective view of the lower jaw member of the scissor system depicted in FIG. 25, in accordance with a seventh embodiment of the present invention;

FIG. 30 illustrates a perspective view of the upper jaw member of the scissor system depicted in FIG. 25, in accordance with a seventh embodiment of the present invention;

FIG. 31 illustrates a perspective view of a seventh alternative scissor system, in accordance with an eighth embodiment of the present invention;

FIG. 32 illustrates a perspective view of the upper jaw member of the scissor system depicted in FIG. 31 with the hollow tube member being shown in phantom for purposes of illustration, in accordance with an eighth embodiment of the present invention;

FIG. 33 illustrates a perspective view of the lower jaw member of the scissor system depicted in FIG. 31, in accordance with an eighth embodiment of the present invention;

FIG. 34 illustrates a perspective view of the upper jaw member of the scissor system depicted in FIG. 31, in accordance with an eighth embodiment of the present invention;

FIG. 35 illustrates a perspective view of the lower jaw member of the scissor system depicted in FIG. 31 with a portion of the hollow tube member removed for purposes of illustration, in accordance with an eighth embodiment of the present invention; and

FIG. 36 illustrates a second perspective view of the lower jaw member of the scissor system depicted in FIG. 31, in accordance with an eighth embodiment of the present invention.

The same reference numerals refer to the same parts throughout the various Figures.

### DETAILED DESCRIPTION OF THE INVENTION

The following description of the preferred embodiment(s) is merely exemplary in nature and is in no way intended to limit the invention, or uses.

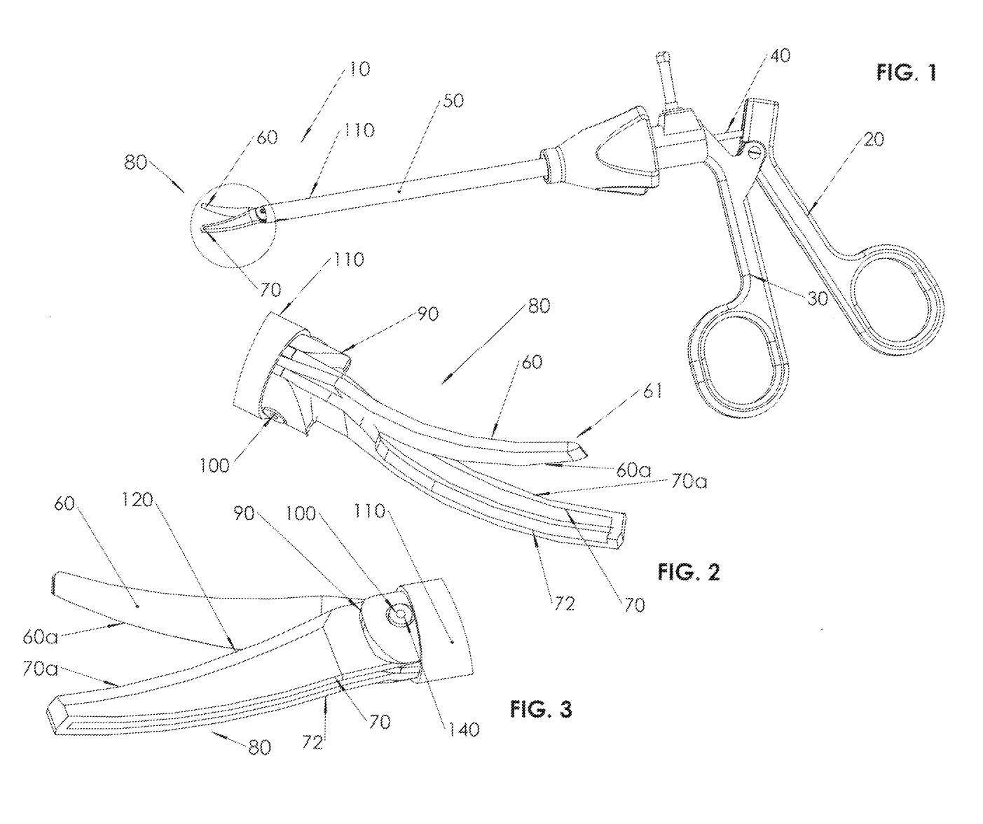

Referring to the Figures generally, and specifically to FIG. 1, there is shown an illustrative endoscopic forceps system generally at 10. It should be appreciated, however, that the scissor systems of the present invention may be used with any type of cutting instrument or system and is not limited in any way to only surgical instruments or systems.

The system 10, primarily includes, at a proximal portion thereof, a pair of handle members 20 and 30, respectively, wherein handle member 20 is free to pivot (e.g., via a pivot point 22) on or about handle member 30. When handle member 20 is caused to pivot on or about handle member 30, e.g., by the action of the surgeon, a reciprocating action (e.g., in a back-and-forth or to-and-fro motion) is effected on an activating shaft member 40 that may be at least partially disposed within a hollow tube member 50. This reciprocating action caused by the opening and/or closing of handle members 20 and 30 ultimately results in the opening apart and/or closing together of blade or jaw members 60 and 70, respectively, of the scissor system 80, which is disposed at a distal portion of the system 10. It is this opening apart and/or closing together of blade or jaw members 60 and 70 that provide the cutting action of the scissor system 80.

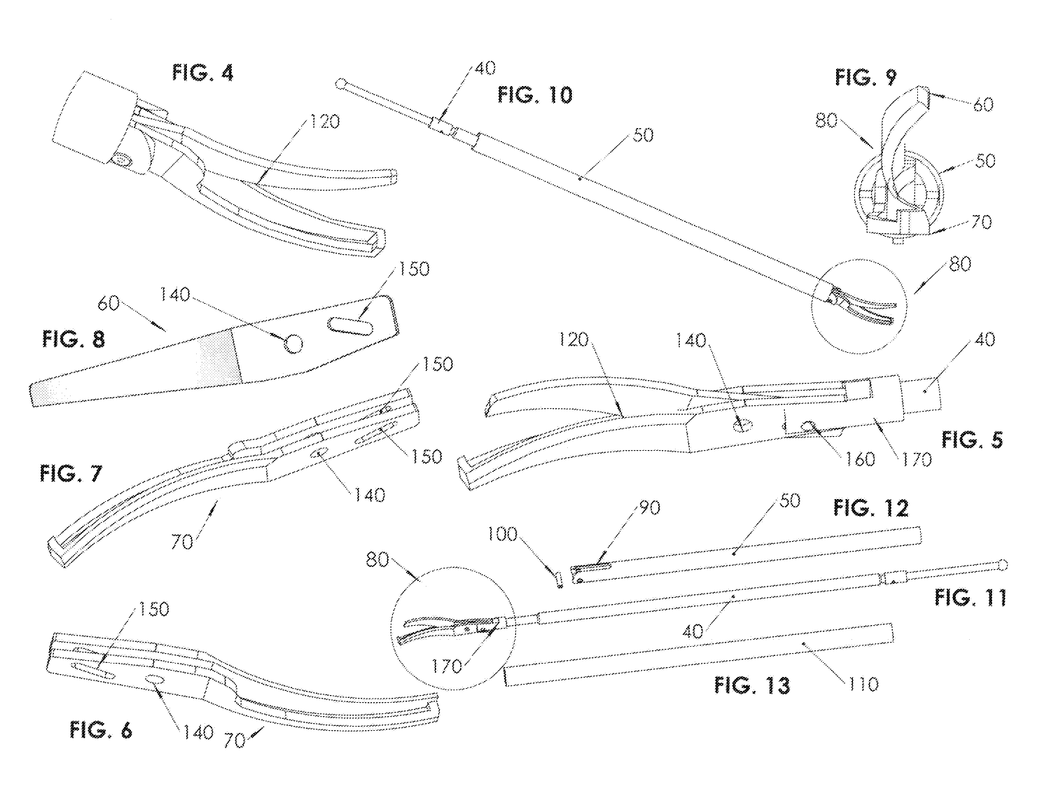

Referring to FIGS. 2-13, there is shown various additional views of the system 10 depicted in FIG. 1. For purposes of reference, the single blade or jaw member 60 is referred herein as the “upper” jaw member and the reinforced blade or jaw member 70 is referred herein as the “lower” jaw member. However, it should be appreciated that these terms are being used for reference purposes only and are not meant to limit the scope of the present invention. Likewise, it should be appreciated that terms such as “top” or “bottom” are also being used for reference purposes only and are not meant to limit the scope of the present invention. Furthermore, for purposes of brevity, the phrase “jaw member, as used herein,” will be understood to also optionally include at least one cutting surface formed thereon.

The jaw members 60 and 70 may be jointly held within a clevis member 90 or other suitable device and may be generally free to rotate on or about a common pin member 100 or other suitable device. Additionally, an isolative material 110 (i.e., heat shrink) may be placed over the tube member 50 so as to avoid undesired arcing between the tube member 50 and any adjacent tissue (e.g., when the system 10 is used in conjunction with electrically powered use during a surgical procedure).

By way of a non-limiting example, as the upper jaw member 60 is rotating towards the lower jaw member 70, an attached secondary beam member 72 maintains the engagement of the cutting edges 60a and 70a of jaw members 60 and 70, respectively, through the entirely or substantially through the entirety of the cutting action and help support the upper jaw member 60 against being pushed apart off of the lower jaw member 70 or skewing therefrom, thus resulting in a failure to cut or a poor quality cut.

By way of a non-limiting example, the secondary beam member 72 may be parallel to the lower jaw member 70 at a distance that is equal to the thickness of the upper jaw member 60 plus a small tolerance factor (e.g., preferably below 0.002 inches or 0.05 mm).

The curvature of the upper jaw member 60 preferably protrudes the curvature of the lower jaw member 70 so that the cutting point propagates from a contact point 120 to the distal end thereof upon closure of the jaw members 60 and 70, respectively. That is, the curvature of the upper jaw member 60 is not identical to the curvature of the lower jaw member 70. By way of a non-limiting example, upper jaw member 60 should conform to the space between the lower jaw member 70 and secondary beam member 72 by elastic deflection.

By way of a non-limiting example, both jaw members 60 and 70 may pivot on the common pin member 100 that may be positioned in an area defining a hole 140. Cams 150, being activated by a common pin member 160, may be held in by a clevis member 170 at the distal end of the activating shaft member 40.

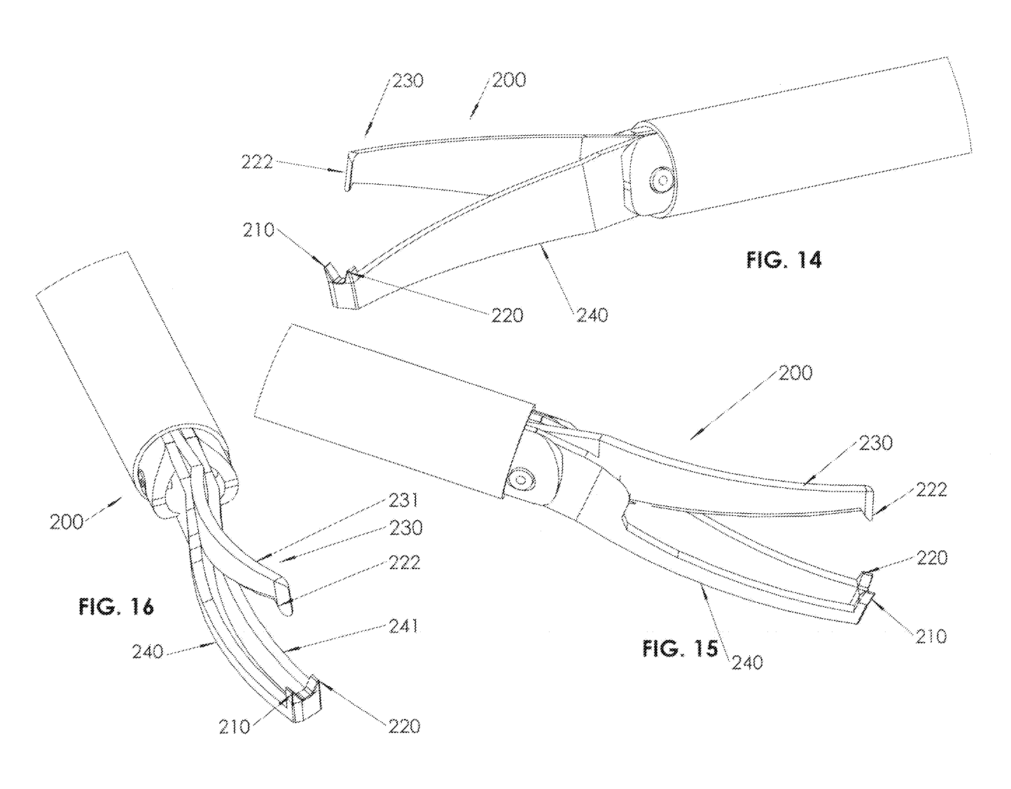

Referring to FIGS. 14-16, there is shown another embodiment of the scissor systems of the present invention. In these views, the scissor system 200 is provided with a least one, and more preferably, a plurality of hook members 210, 220, 222, respectively, located at the distal portions of the jaw members 230, 240, respectively. It should also be appreciated that the jaw members 230, 240, respectively, can be “hookless” as well, include only one hook member each, and/or two hook members each, or any combination thereof. By way of a non-limiting example, this is feature is particularly advantageous when attempting to anchor slippery tissue during a surgical procedure. As previously noted, the jaw members 230, 240, respectively, may include cutting edges formed on a surface thereof.

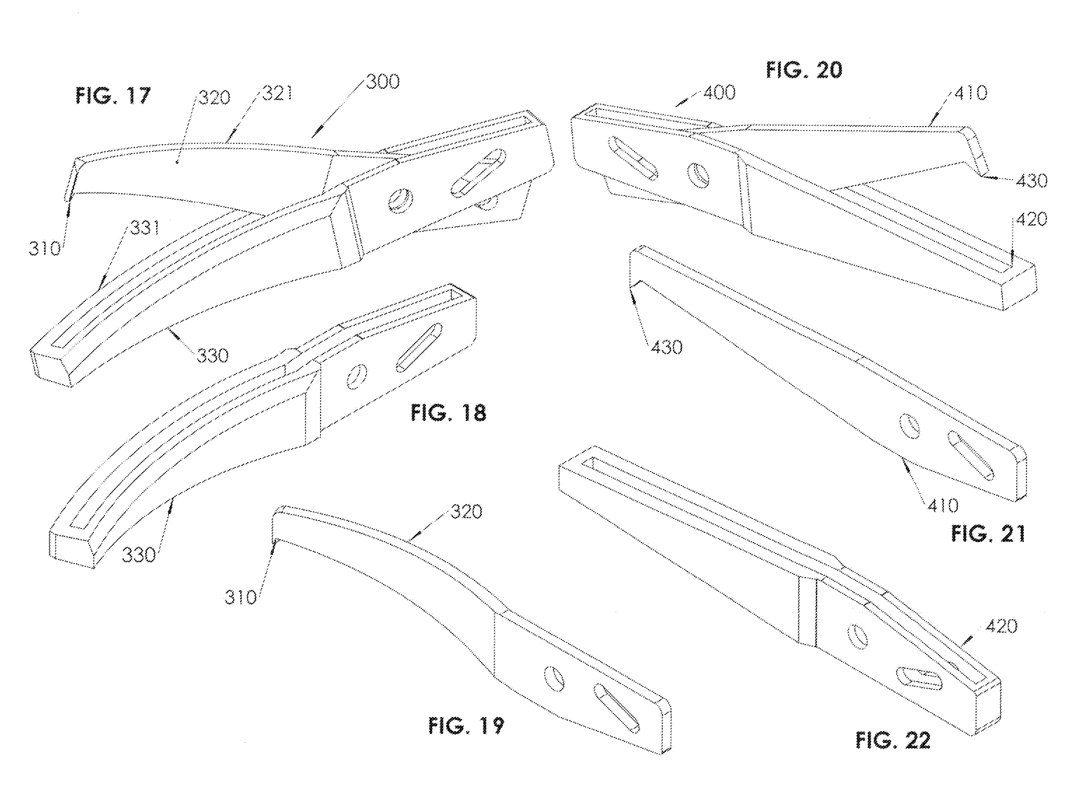

Referring to FIGS. 17-19, there is shown another alternative embodiment wherein the scissor system 300 is shown with a single hook member 310 located on the “top” or “upper” jaw member 320, whereas the “lower” or “bottom” jaw member 330 does not include a hook member. Again, it should also be appreciated that the jaw members 320, 330, respectively, can be “hookless” as well, include only one hook member each, and/or two hook members each, or any combination thereof. As previously noted, the jaw members 320, 330, respectively, may include cutting edges formed on a surface thereof.

Referring to FIGS. 20-22, there is shown still another alternative embodiment wherein the scissor system 400 does not includes any curved jaw members, but rather includes two substantially straight jaw members 410, 420, respectively. In this view, a single hook member 430 is located on the “top” or “upper” jaw member 410, whereas the “lower” or “bottom” jaw member 420 does not include a hook member. Again, it should also be appreciated that the jaw members 410, 420, respectively, can be “hookless” as well, include only one hook member each, and/or two hook members each, or any combination thereof. As previously noted, the jaw members 410, 420, respectively, may include cutting edges formed on a surface thereof.

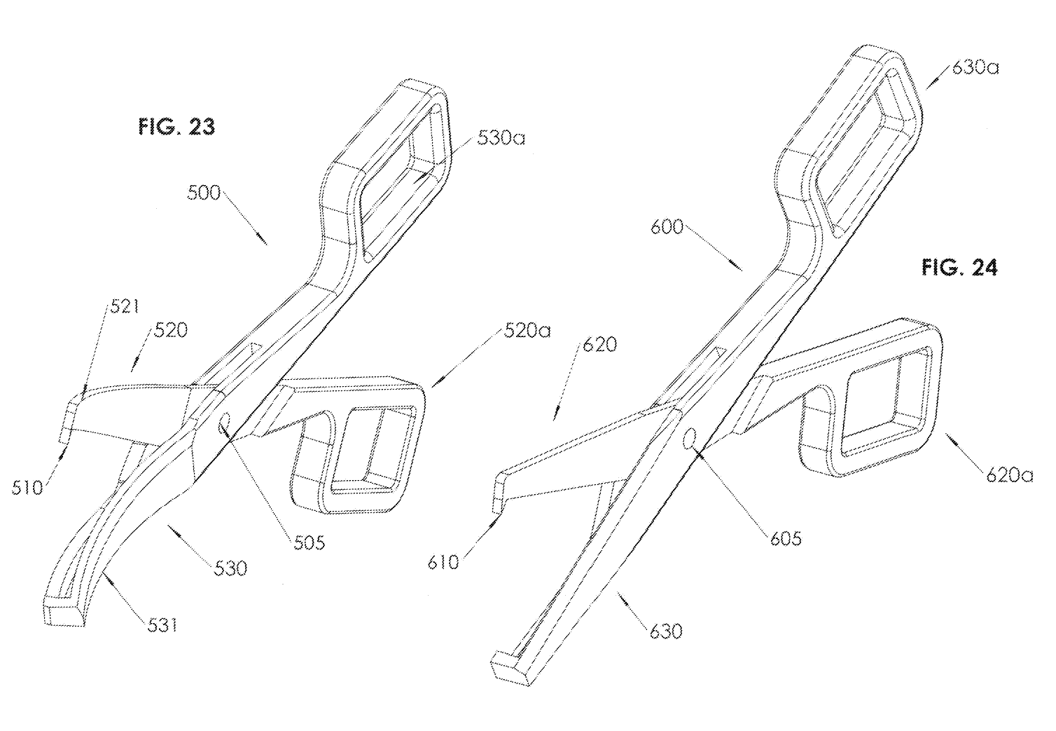

Referring to FIG. 23, there is shown a further alternative embodiment, wherein a general purpose, curved scissor system 500 is provided, wherein the scissor system 500 is shown with a single hook member 510 located on the “top” or “upper” jaw member 520, whereas the “lower” or “bottom” jaw member 530 does not include a hook member. Again, it should also be appreciated that the jaw members 520, 530, respectively, can be “hookless” as well, include only one hook member each, and/or two hook members each, or any combination thereof. As previously noted, the jaw members 520, 530, respectively, may include cutting edges formed on a surface thereof.

The jaw members 520, 530, respectively, may be provided with handle members 520a, 530a, respectively, that may pivot relative to one another at a pivot point 505. In this view, the jaw members 520, 530, respectively, may be directly joined to, or integrally formed with, the handle members 520a, 530a, respectively.

Referring to FIG. 24, there is shown still a further embodiment, wherein a general purpose, substantially straight scissor system 600 is provided, wherein the scissor system 600 is shown with a single hook member 610 located on the “top” or “upper” jaw member 620, whereas the “lower” or “bottom” jaw member 630 does not include a hook member. Again, it should also be appreciated that the jaw members 620, 630, respectively, can be “hookless” as well, include only one hook member each, and/or two hook members each, or any combination thereof. As previously noted, the jaw members 620, 630, respectively, may include cutting edges formed on a surface thereof.

The jaw members 620, 630, respectively, may be provided with handle members 620a, 630a, respectively, that may pivot relative to one another at a pivot point 605. In this view, the jaw members 620, 630, respectively, may be directly joined to, or integrally formed with, the handle members 620a, 630a, respectively.

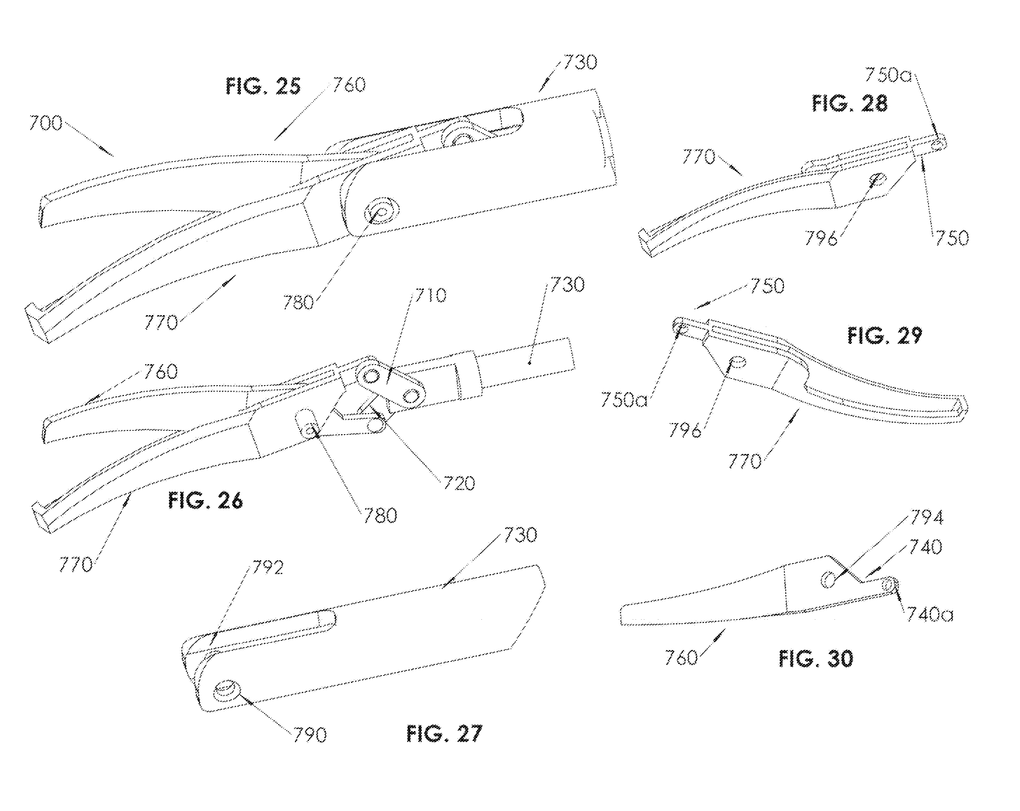

Referring to FIGS. 25-30, there is shown still yet another further embodiment, wherein a scissor system 700 is provided. The system 700 may be activated (i.e., in a cutting action) by one or more link members 710, 720, respectively, that may be pivotally attached to a distal end of an activating shaft member 730 via holed or keyed male ends or protuberances 740, 750, respectively, (see areas defining holes at 740a, 750a, respectively), formed on the proximal portions of the jaw members, 760, 770, respectively. As previously noted, the jaw members 760, 770, respectively, may include cutting edges formed on a surface thereof.

The jaw members, 760, 770, respectively, can be joined to the distal end of the activating shaft member 730 via a common pin member 780 (e.g., aligned through an area defining one or more holes 790, 792, respectively formed in the distal portion of the activating shaft member 730, and an area defining a hole 794, 796, respectively, formed on the jaw members, 760, 770, respectively), such that, as the activating shaft member 730 moves in a back-and-forth or to-and-fro motion (due to the actuation of the handle members (not shown)), the jaw members, 760, 770, respectively, will open or close, as the case may be, and in doing so, will be selectively operable to perform a cutting action.

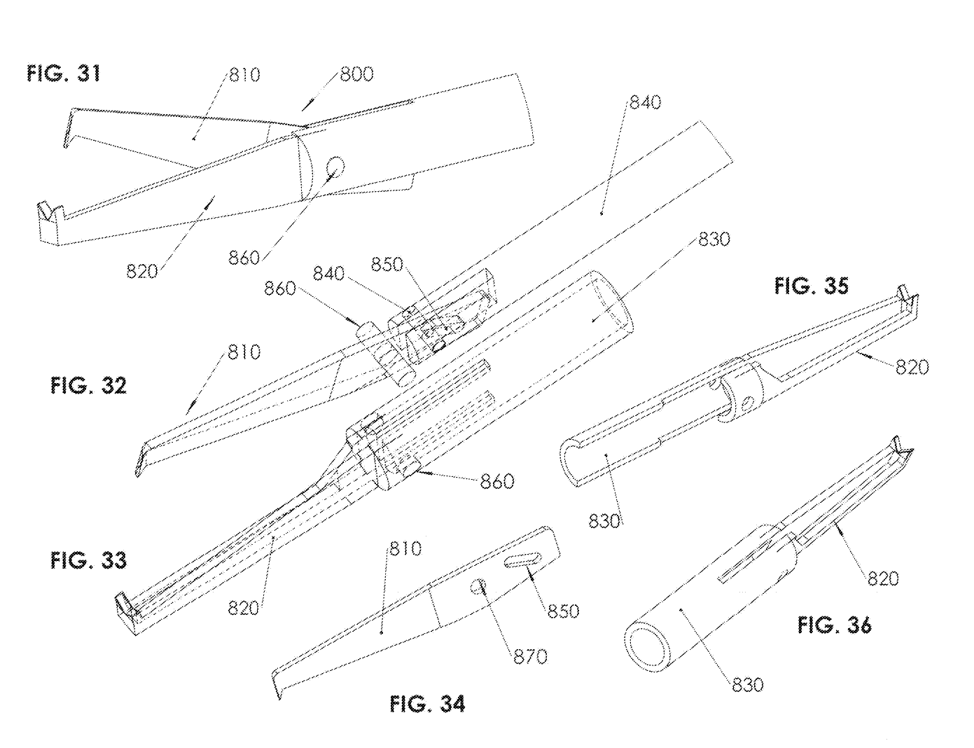

Referring to FIGS. 31-36, there is shown still yet another further alternative embodiment, wherein a scissor system 800 is provided. The system 800 includes a substantially straight movable “top” or “upper” jaw member 810 and a “static” or “fixed,” “bottom” or “lower” jaw member 820. As previously noted, the jaw members 810, 820, respectively, may include cutting edges formed on a surface thereof.

It should also be appreciated that one or more curved jaw members may also be used in conjunction with this embodiment as well.

The lower jaw member 820 may be preferably integrally formed with the distal end of a tube member 830 or may be joined thereto in any number of suitable manners (e.g., adhesively joined, welded, and/or the like). An activating shaft member 730 may reciprocate within the tube member 830 and thus activate jaw member 810 via a pin member 840 sliding within a cam 850 formed in jaw member 810. Jaw member 810 may pivot on or about the jaw member 820 via common pin member 860 (e.g., similar to the arrangement described in connection with FIGS. 24-29) disposed within an area defining a hole 870 formed in jaw member 810.

The variants shown herein are for illustrative purposes only. It should be appreciated that features such as but not limited to hooks, cross supports between jaws and secondary beams, curvature changes, and additional features to the blades, such as but not limited to rasped edges, scallops, and/or the like, may be added to the scissor systems of the present invention.

Furthermore, although the above description pertains primarily to endoscopic scissor systems, it should be appreciated that the present invention is applicable as well to open surgery scissors, industrial scissors such as those used by barbers, tailors, and other craftsmen, as well as large scissors-like applications such as logging and shearing of substances with two blades.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A scissor system, comprising:
a first jaw member having a cutting surface formed thereon;
a second jaw member having a cutting surface formed thereon; and
a beam member positioned substantially parallel to the cutting surface of the second jaw member.

2. The scissor system according to claim 1, wherein the first and second jaw members are pivotally engaged to each other so as to be selectively operable to open apart from each other and close together towards each other to selectively perform a cutting action.

3. The scissor system according to claim 2, wherein the beam member maintains engagement of the cutting surfaces of the first and second jaw members through substantially the entirety of the cutting action.

4. The scissor system according to claim 3, wherein the beam member supports the first jaw member so as to substantially prevent the first jaw member from being pushed away from the second jaw member during the cutting action.

5. The scissor system according to claim 1, wherein either the first or second jaw members include a curved portion.

6. The scissor system according to claim 5, wherein the curved portions of the first and second jaw members are not identical.

7. The scissor system according to claim 1, wherein the first jaw member includes a hook member formed on a distal portion thereof.

8. The scissor system according to claim 1, wherein the second jaw member includes a pair of spaced and opposed hook members formed on a distal portion thereof.

9. The scissor system according to claim 1, wherein the second jaw member and the beam member define a cavity formed therebetween.

10. The scissor system according to claim 9, wherein the first jaw member is selectively operable to be at least partially received within the cavity.

11. A scissor system, comprising:
a first jaw member having a cutting surface formed thereon;
a second jaw member having a cutting surface formed thereon; and
a beam member positioned substantially parallel to the cutting surface of the second jaw member;
wherein the first and second jaw members are pivotally engaged to each other so as to be selectively operable to open apart from each other and close together towards each other to selectively perform a cutting action;
wherein the beam member maintains engagement of the cutting surfaces of the first and second jaw members through substantially the entirety of the cutting action.

12. The scissor system according to claim 11, wherein the beam member supports the first jaw member so as to substantially prevent the first jaw member from being pushed away from the second jaw member during the cutting action.

13. The scissor system according to claim 11, wherein either the first or second jaw members include a curved portion.

14. The scissor system according to claim 13, wherein the curved portions of the first and second jaw members are not identical.

15. The scissor system according to claim 11, wherein the first jaw member includes a hook member formed on a distal portion thereof.

16. The scissor system according to claim 11, wherein the second jaw member includes a pair of spaced and opposed hook members formed on a distal portion thereof.

17. The scissor system according to claim 11, wherein the second jaw member and the beam member define a cavity formed therebetween.

18. The scissor system according to claim 17, wherein the first jaw member is selectively operable to be at least partially received within the cavity.

19. A scissor system, comprising:
a first jaw member having a cutting surface formed thereon;
a second jaw member having a cutting surface formed thereon; and
a beam member positioned substantially parallel to the cutting surface of the second jaw member;
wherein the first and second jaw members are pivotally engaged to each other so as to be selectively operable to open apart from each other and close together towards each other to selectively perform a cutting action;
wherein the beam member maintains engagement of the cutting surfaces of the first and second jaw members through substantially the entirety of the cutting action;
wherein the beam member supports the first jaw member so as to substantially prevent the first jaw member from being pushed away from the second jaw member during the cutting action.

20. The scissor system according to claim 19, wherein either the first or second jaw members include a curved portion.

21. The scissor system according to claim 20, wherein the curved portions of the first and second jaw members are not identical.

22. The scissor system according to claim 19, wherein the first jaw member includes a hook member formed on a distal portion thereof.

23. The scissor system according to claim 19, wherein the second jaw member includes a pair of spaced and opposed hook members formed on a distal portion thereof.

24. The scissor system according to claim 19, wherein the second jaw member and the beam member define a cavity formed therebetween.

25. The scissor system according to claim 24, wherein the first jaw member is selectively operable to be at least partially received within the cavity.
